(19) United States
(12) Patent Application Publication
(10) Pub. No.: US 2022/0013219 A1
(43) Pub. Date: Jan. 13, 2022

(54) COMPUTER-IMPLEMENTED METHOD AND RELATED SYSTEM FOR PROVIDING CLEAR ALIGNERS DIRECTLY TO PATIENTS

(71) Applicant: STRAIGHT TEETH SOLUTIONS, INC., Palm City, FL (US)

(72) Inventors: Bruce Goldblatt, Palm City, FL (US); Ivana Sears, Palm City, FL (US); Dimitri Constantine, Palm City, FL (US)

(73) Assignee: STRAIGHT TEETH SOLUTIONS, INC., Palm City, FL (US)

(21) Appl. No.: 17/369,779

(22) Filed: Jul. 7, 2021

Related U.S. Application Data
(60) Provisional application No. 63/048,932, filed on Jul. 7, 2020.

(51) Int. Cl. G16H 40/20; G16H 30/20; G16H 10/60; G06Q 20/12; G06Q 30/00; G06Q 10/10; G06Q 40/08; G06Q 40/02; G06Q 20/02; G16H 40/67; G06F 16/9537; A61C 7/00; A61C 7/08; A61C 9/00

(52) U.S. Cl. CPC G16H 40/20; G06Q 50/04; G16H 10/60; G06Q 20/127; G06Q 30/018; G06Q 10/1095; G06Q 40/08; G06Q 40/025; G06Q 20/023; G16H 40/67; G06F 16/9537; A61C 7/002; A61C 7/08; A61C 9/004; G16H 30/20

(57) ABSTRACT

An integrated technological solution for providing, fitting, and treating a patient with clear aligners by employing specially programmed computing devices and systems, including a computer-implemented software application accessible by a user on an electronic device.

### CROSS-REFERENCE TO RELATED APPLICATIONS

This application is a non-provisional of, and claims priority to, U.S. Patent Application Ser. No. 63/048,932, “Computer-Implemented Method and Related System for Providing Clear Aligners Directly to Patients,” filed on Jul. 7, 2020, which application is incorporated herein in its entirety.

### FIELD OF THE INVENTION

The present invention generally relates to software and business methods, and specifically, to a unique method, including a computer-implemented method and associated technology, that enables patients to receive clear aligner dental solutions to patients with the cost savings and ease of a direct to consumer model, but with the care and supervision of a doctor.

### BACKGROUND OF THE INVENTION

Currently, patients that are desiring to obtain clear aligners through a do-it-yourself (“DIY”) direct to consumer (“DTC”) model have the option to engage with one of multiple “clubs” that provide these clear aligners. Primarily, these patients are looking for a lower price and a more convenient solution. Patients look for a DTC solution for three primary reasons, in no particular order (1): it can be more affordable compared to in office, clear aligner treatment from well-known brands; (2) it is convenient, as “do it yourself” kits are mailed directly to the patients' home and have no immediate doctor oversight with the patient; and (3) the large providers have created this DTC market and aggressively advertise to consumers that seek to have their teeth straightened to improve their look, confidence, or career mobility. These DIY “clubs” rely on imaging or 3D scanning conducted by non-medical personnel—often at shopping mall kiosks—and do not involve a doctor for exams, x-rays or scans, fittings, and ongoing treatments. There is a need, therefore, for a method and system of delivering clear aligner dental solutions to patients with the cost savings and ease of a direct to consumer model, but with the care and supervision of a doctor. Existing Direct to Consumer models do not allow for attachments or inter proximal reductions (IPR) that greatly improve the success of clear aligners. Often, this leads to patients who receive less than optimal results and risk aggravation of other conditions when a dentist is not involved in the process. In some instances, dentists and dental offices are not prepared to provide clear aligners as an option for their patients. In these cases, the dentist may end up losing the patient to another office that does offer the clear aligners, or, worse, the patient may end up with a DIY solution and less than optimal results.

There is a need, therefore, for a method of delivering clear aligner dental solutions to patients with the cost savings and ease of a direct to consumer model, but with the care and supervision of a doctor, where the method further provides an improvement to the functionality of the computer system through which it is implemented.

### SUMMARY OF THE INVENTION

We disclose, therefore, a unique method and system that provides an improvement over the current state of the art, including improvements to computer-implemented software applications and associated technology, that provides clear aligners directly to patients along with doctor-supervised treatment, and also provides doctors with patient referrals, costs savings by eliminating and simplifying practice overhead for doctors by providing marketing and referral of patients, and administrative management through prequalification by confirming insurance, or providing third party financing and credit card payment options and related administration, so the doctor is free to focus on the exam, fitting and follow up visits with the patient.

Embodiments of the invention also provide a direct technological portal for the doctor to upload to a remote database any required exams, x-rays, 3D scans and photos, to a treatment planner, and on selection by patient and doctor automatically submitted to manufacturer for custom aligners for the treatment plan based on patient's budget and expectations.

Additionally, embodiments of the invention handle the marketing for dentists providing referrals of pre-qualified (interested in aligners and ability to pay) patients only with doctor supervised treatment, and also proves the same to dentists, handle confirming insurance, arranging financing if needed, and handle patient scheduling and follow-up. Embodiments of the computer-implemented method interface with practice management software in order to handle appointment scheduling, progress of manufacturing and shipment of aligners, patient satisfaction, and payment to dentist of fees.

Briefly, according to one embodiment of the present disclosure, we disclose a computer-implemented method of providing, fitting, and treating a patient with clear aligners, that employs specially programmed computing devices and systems and includes the steps or acts of: providing a computer-implemented software application (the “App”) accessible by a user on an electronic device comprising at least a processor and a memory; identification by the App of the user's geographic location by, for example, querying of a GPS module in communication with the device; querying of a database of available providers (for example dentists or orthodontists) and returning a display of available providers to the user that meet required location parameters; interfacing by the App with the provider's practice management software in order to return available appointment dates and times; coordination of scheduling of an appointment between the user and the provider via the App; confirmation via the App of other parameters (such as insurance, demographic information, payment information, etc. . . . ); providing via the App appointment confirmation and reminders to the user; once the user has had the consultation with the provider, the provider, via a provider-side interface to a backend database of the invention, uploads pertinent data such as, but not limited to intraoral 3D scans, x-rays, photographs, and other information pertinent to the user/patient; the application system, upon confirmation by the provider, provides the uploaded data to a manufacturer; and the manufacturer, accessing the system, utilizes the uploaded data to create the clear aligners and sends them directly to the Provider/doctor to be delivered to the patient; whereby the patient receives customized clear aligners and a treatment plan with the convenience of a direct to consumer DIY model but with the oversight of a dentist or orthodontist.

A revenue model is included as part of the invention. Revenue is derived through, for example, a subscription model where a provider may pay for a level of service and, in return, receive marketing, referrals, hardware, and custom-programmed treatment tools. The software and computing aspects of the invention may be realized as a custom, or localized, installation or, alternatively, in a software-as-a-service model where the user and/or provider interacts with the various aspects of the invention via a wide area network, such as the Internet.

### BRIEF DESCRIPTION OF THE DRAWINGS

In the accompanying figures, like reference numerals refer to identical or functionally similar elements throughout the separate views. The accompanying figures, together with the detailed description below are incorporated in and form part of the specification and serve to further illustrate various embodiments and to explain various principles and advantages all in accordance with the present invention, in which:

FIG. 1 shows a key for the flowchart symbols used in FIG. 2A, FIG. 2B, FIG. 2C, FIG. 2D, and FIG. 2E;

FIG. 2A, FIG. 2B, FIG. 2C, FIG. 2D, and FIG. 2E are operational flow diagrams of an application flow, according to an embodiment;

FIG. 3 is an informational graphic displaying features and pricing of treatments utilizing an embodiment of the invention; and

FIG. 4 is an informational graphic displaying revenue models utilizing an embodiment of the invention.

While the invention as claimed can be modified into alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the scope of the present invention.

### DETAILED DESCRIPTION

Before describing in detail embodiments that are in accordance with the present invention, it should be observed that the embodiments reside primarily in combinations of method steps and system components related to systems and methods for placing computation inside a communication network. Accordingly, the system components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein. Thus, it will be appreciated that for simplicity and clarity of illustration, common and well-understood elements that are useful or necessary in a commercially feasible embodiment may not be depicted in order to facilitate a less obstructed view of these various embodiments.

In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

We describe a system, method, and service that (1) identifies and attracts interested patients, (2) efficiently matches patients with their local doctor, (3) handles the process via a network-connected application, and (4) requires that a doctor see, treat and administer the aligners with the patient. In this way, through this type of system and method that provides for and creates an improvement in the functionality of the computer running the network-connected application, the patient is provided with a more favorable and healthy outcome.

In the method, system, and service as described herein, dentists and dental professionals are able to receive referrals of pre-qualified (interested in aligners and ability to pay) patients. The disclosure describes an integrated technological solution for dental practices that provides for an improvement to the computer system and overall integrated technical solution that handles confirming insurance, arranging financing if needed, and handling patient scheduling and follow-up. We provide a solution, currently lacking in the art, to interface with a dentist's practice management software to handle appointment scheduling, progress of manufacturing and shipment of aligners, patient satisfaction, and payment to dentist of fees.

The method.

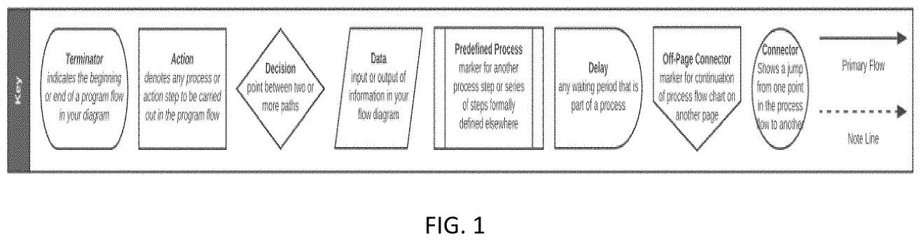

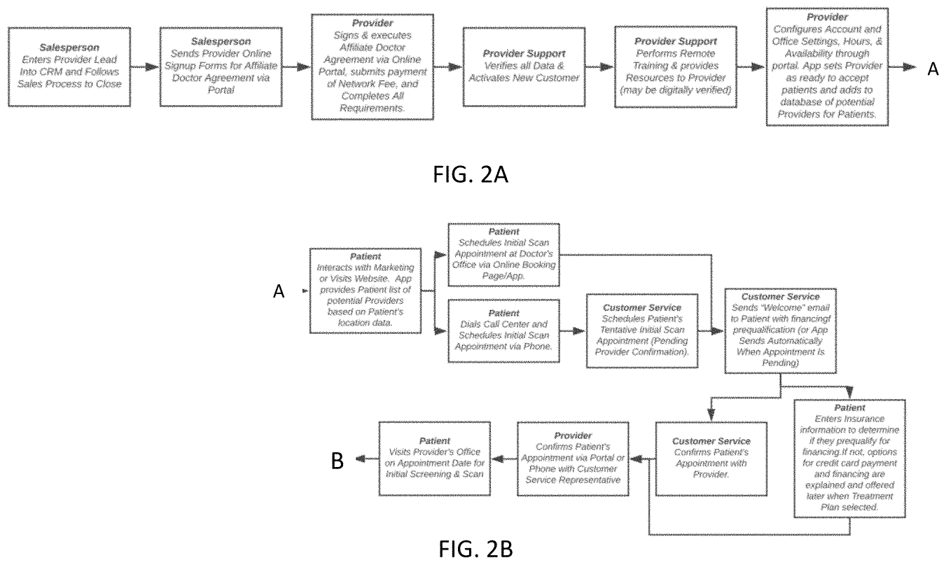

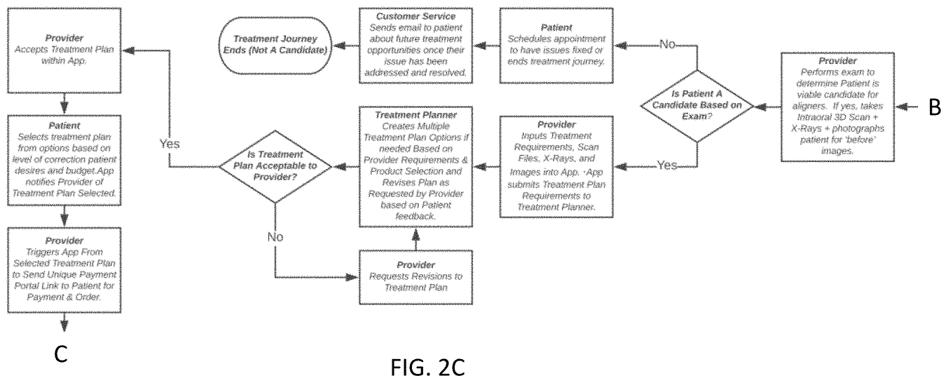

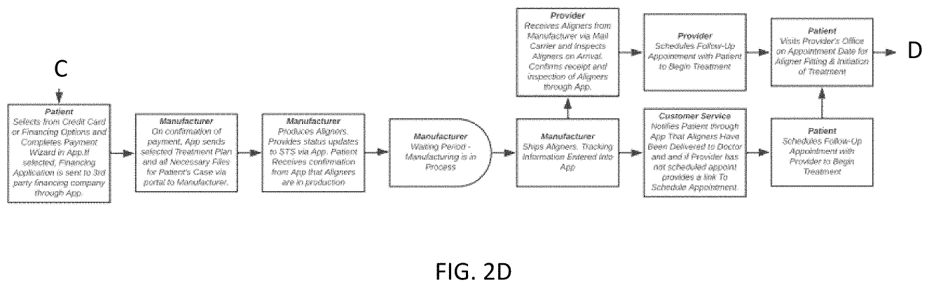

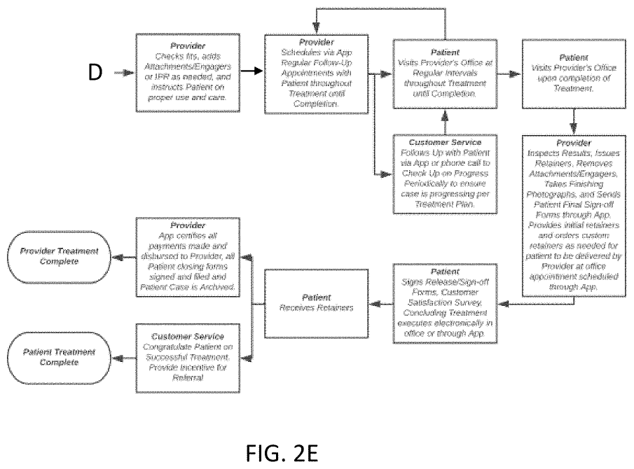

Referring now to the application flow diagrams shown in FIG. 2A through FIG. 2E (using the key of FIG. 1), we discuss method steps involved in a preferred embodiment of the present invention. As used herein, the Service coordinates the flow of steps between a patient, the provider, and the manufacturer utilizing a computer-implemented method as described herein on a system including physical computing hardware, servers, and communications networks. A provider is a dentist or orthodontist that has been pre-qualified and signed-up to be an affiliate of the Service in an approved network of providers. The Salesperson is part of the Service and may be a human person utilizing the computer-implemented method, or may be accomplished as a discrete module of the computer-implemented method. As used herein, Salesperson, Provider Support, Treatment Planner and Customer Service, are integrated functions/modules of the Service. Manufacturer and Financing Company may be integrated with the Service, or may be third party providers to the Service.

In a preferred embodiment of the invention, a user interacts with hardware systems and programmed modules to accomplish the method steps, the method steps of FIG. 2A include:

Salesperson Enters Provider Lead Into CRM and Follows Sales Process to Close;

Salesperson Sends Provider Online Signup Forms for Affiliate Doctor Agreement via Portal;

Provider Signs & executes Affiliate Doctor Agreement via Online Portal, submits payment of Network Fee, and Completes All Requirements. App will send annual renewal reminder and process payment of Network Fee;

Provider Support Verifies all Data & Activates New Customer;

Provider Support Performs Remote Training & provides Resources to Provider (may be digitally verified);

Provider Configures Account and Office Settings, Hours, & Availability through portal. App sets Provider as ready to accept patients and adds to database of potential Providers for Patients.

In FIG. 2B, Patient Interacts with Marketing or Visits Website. App provides Patient list of potential Providers based on Patient's location data;

Patient Schedules Initial Scan Appointment at Doctor's Office via Online Booking Page/App. App notifies Patient immediately that appointment will be confirmed within a specified time frame, such as 24 hours, and provides Patient Informed Consent and Patient release to comply with HIPAA. App will link with Doctor's Practice Management System for scheduling of appointments. App coordinates with selected Provider's calendar and enters appointment in Provider's calendar and confirms appointment with Patient;

Alternatively, Patient Dials Call Center through App and Schedules Initial Scan via Phone;

Customer Service Schedules Patient's Tentative Initial Scan Appointment (Pending Provider Confirmation);

Customer Service or App sends “Welcome” email with financing prequalification and let's patient know appointment is pending confirmation by Provider. Alternatively, the App will generate “Welcome Intro” email;

Patient confirms with their insurance company to determine if they prequalify for insurance coverage. Customer Service assists Patient with forms to file their claim and handling of reimbursements directly with insurance company after purchase of the aligners. If not, options for credit card payment and financing are explained and offered later when Treatment Plan selected. The App offers a prequalification link to third-party financing and Patient can enter estimated financing amount and finance company does a ‘soft pull’ credit check which does not affect Patient's credit score, to provide a payment estimate subject to credit and APR;

Customer Service Customer Service calls each provider to confirm dates and times requested. The information is then given to the doctor's office per the patient's questionnaire and personal info submission in the booking system. Confirms Patient's Appointment with Provider through app or phone call. App alerts Customer Service for all new appointment requests daily. In embodiments this is handled by App by connecting with Provider's Practice Management System—setting appointments and notifying Customers;

Provider Confirms Patient's Appointment via Portal or Phone call with Customer Service Representative. App sends Booking Confirmation email and Patient visits Provider's Office on Appointment Date for Initial Screening & Scan. App sends a reminder email, for example, 12 hours prior to appointment;

As shown in FIG. 2C, Provider performs exam to determine Patient is viable candidate for aligners. If yes, takes intraoral 3D Scan+X-Rays+photographs patient for ‘before’ images. Doctor discusses additional services IPR (Inter Proximal Reduction), attachments, etc. . . . that are needed for proper aligner treatment and satisfactory results;

Steps dependent on Outcome of Provider exam:
a) If Patient does not pass exam OR patient simply is not a candidate due to age or other matters, then Customer Service sends email to patient about future treatment opportunities once their issue has been addressed and resolved.
b) If Patient cancels/no show, then App/Customer Service sends “What happened?” email to patient and offers a second consultation booking with a link to booking system attached.
c) If Patient passes exam, then in the Provider's office, if doctor and patient agree, a follow up Appointment is scheduled to review treatment plan and package selection. If patient selects ‘Complete Solution’ option which is most comprehensive treatment plan a follow up appointment is not needed. The new appointment date and time is directly loaded into Provider's Practice Management System/Calendar.

Provider Inputs Treatment Requirements, Scan Files, X-Rays, and Images into App. App submits Treatment Plan Requirements to Treatment Planner;

Treatment Planner Creates Multiple Treatment Plan Options if needed Based on Provider Requirements & Product Selection and Revises Plan as Requested by Provider based on Patient feedback;

Provider Accepts Treatment Plan Options (Move Forward to Order)—OR—requests revisions to Treatment Plan (Send revisions to Treatment Planner);

Patient Selects treatment plan from options based on level of correction patient desires and budget. App notifies Provider of Treatment Plan Selected; and

Provider initiates APP to send Unique Payment Portal Link to Patient.

As shown in FIG. 2D, Patient Selects from Credit Card or Financing Options and Completes Payment Wizard in App. If selected, Financing Application is sent to 3rd party financing company through App. Finance company is directly connected with the App for application submission and decision and all required notices required for financing and finance company provides decision on credit application within 60 seconds;

Financing Company notifies patient directly through App of financing decision. If approved, Patient is sent “Congratulations” email through App confirming financing approval. If not approved, Customer Service works with Patient for alternative form of payment;

Manufacturer On confirmation of payment, App sends selected Treatment Plan and all Necessary Files for Patient's Case via portal to manufacturer;

Manufacturer Produces Aligners. Provides status updates via App;

Patient Receives confirmation from App that Aligners are in production;

Manufacturer Ships Aligners. Tracking Information Entered Into App and sent to Provider and Patient;

Provider Receives Aligners from Manufacturer and Inspects Aligners on Arrival;

Provider Confirms receipt and inspection of Aligners through App. Provider schedules appointment with Patient;

Customer Service Patient notified through App that Aligners Have Been Delivered to Doctor and if Provider has not scheduled appoint provides a link To Schedule Appointment; and

Patient Visits Provider's Office on Appointment Date for Aligner Fitting & Initiation of Treatment.

As shown in FIG. 2E, Provider checks fits, adds Attachments/Engagers or IPR as needed, and instructs Patient on proper use and care;

On receipt of funds from financing company, merchant bank for credit card payment, or PayPal payment, initiates Initial Payment via ACH to Provider through App;

Provider Schedules via App Regular Follow-Up Appointments with Patient throughout Treatment until Completion;

Patient Visits Provider's Office at Regular Intervals throughout Treatment until Completion as scheduled through App. Patient treatment is monitored and supervised by the Provider (medical professional) to ensure teeth in both arches straight and that teeth and both arches are in the proper occlusion or the best occlusal position possible for patient;

Customer Service Follows Up with Patient via App or phone call to Check Up on Progress Periodically to ensure case is progressing per Treatment Plan;

Patient Visits Provider's Office upon completion of orthodontic treatment;

Provider Inspects Results, Issues Retainers, Removes Attachments/Engagers, Takes Finishing Photographs, and Sends Patient Final Sign-off Forms through App. Provides initial retainers and orders custom retainers as needed for patient to be delivered by Provider at office appointment scheduled through App;

Patient Signs Release/Sign-off Forms, Customer Satisfaction Survey, Concluding Treatment executes electronically in office or through App;

Patient Receives Retainers;

Customer Service Congratulate Patient on Successful Treatment. Provide Incentive for Referral;

whereby, Patient Treatment is now Complete;

Initiate payment of remaining balance of payment to Provider through App; and

Provider App certifies all payments made and disbursed to Provider, all Patient closing forms signed and filed and then archives Patient Case. Case remains available for access and follow up by Provider and Service; whereby, Provider Treatment is now Complete.

The service.

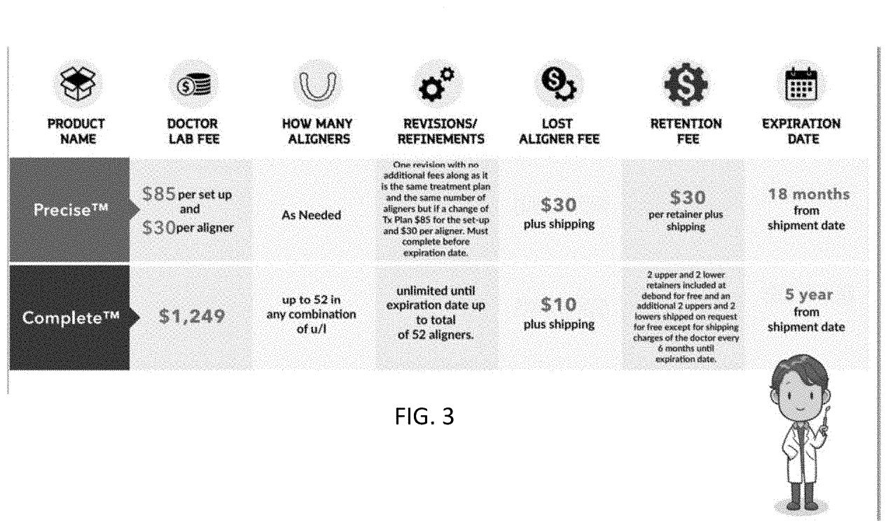

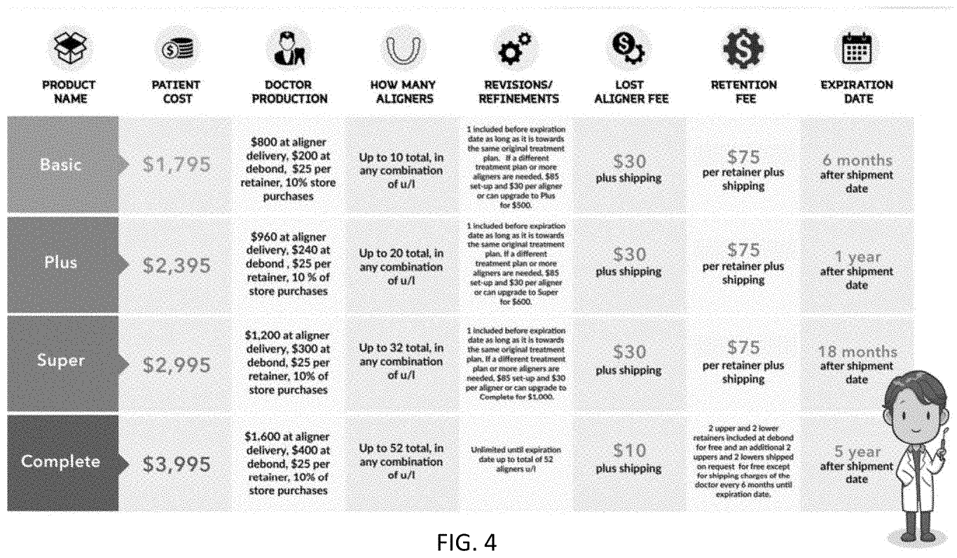

The method of providing clear aligners to patients, as described herein, is preferably provided to users, and specifically the Providers, as a service (hereinafter, the “service”). Revenue models for the service may include software-as-a-service subscription models, pay-as-you-go models, or one-time fee models. We refer to FIGS. 3 and 4 for examples of revenue models related to the service. Users that register and pay for the service receive software modules that provide special programming for the client-provided computing devices. Users of the service also may receive the intraoral scanning hardware, on a loan, lease, or purchase basis.

The system.

Embodiments of the invention include a system in which the clear aligner delivery method system architecture can be implemented. The system includes, at least, a hosting zone, server, a client device, database coupled with the server, and an application gateway facilitating communication between the client device and the server. The client device can be any network-connected computing device such as a cell phone, tablet, personal digital assistant, and the like. The client device may also, in embodiments, be a desktop or laptop computer.

The system can be implemented as follows. A Provider interested in providing clear aligners to patients joins the service. Joining the service can include registering, signing up, and/or or logging in. A Patient can log in as a registered user or “guest” user.

Once registered and signed in, the user (whether the provider or the patient) accesses one or more modules of the system that carry out one or more of the method steps described, above.

From a user's perspective, and in an exemplary and non-limiting embodiment, the method is implemented utilizing both a one or more specially-programmed computing device and digital intraoral scanning hardware. By way of example, and not limitation, the patient may use a mobile phone connected to the internet to access a provided website. The website would prompt the user for various registration details, validate payment, and provide a way to login and create a profile. Alternatively, the website may prompt the user to download and install a software application that, upon installation, makes the user device a specially-programmed computer. In this case, the software application has access to the various hardware modules of the user's device, such as, but not limited to, the GPS and communications networks.

Software modules provide a user interface to guide the user through the process steps. Additionally, one or more software modules may be configured to access, modify, and write to, a one or more database. Based upon user inputs, the software module may transform one or more atoms of data in the database.

The system described above is only one example of a suitable system and is not intended to limit the scope of use or functionality of embodiments of the present invention described above. The system is operational with numerous other general purpose or special purpose computing system environments or configurations. Examples of well-known computing systems, environments, and/or configurations that may be suitable for use with the information processing system include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, clusters, and distributed cloud computing environments that include any of the above systems or devices, and the like. Once programmed to perform the method steps utilizing the modules described in the invention disclosed, general-purpose computing systems become specially-programmed to perform said method steps.

The system may be described in the general context of computer-executable instructions, being executed by a computer system. The system may be practiced in various computing environments such as conventional and distributed computing environments where tasks are performed by remote processing devices that are linked through a communications network. In a distributed computing environment, program modules may be located in both local and remote computer system storage media including memory storage devices.

The system is comprised of client-side and server-side hardware components that communicate with each other over a data network in order to execute the method steps, according to an embodiment of the disclosure. Furthermore, the client-side may be further broken down into patient-side, provider-side, and service-side hardware components.

The client device may represent any type of mobile programmable electronic device, such as a smartphone, wearable computing device such as the Apple Watch®, tablet computer, personal digital assistant, laptop computer, and the like. The client device can include a processing device coupled with memory, storage, and in-device features such as GPS, all operatively linked by bus. One with knowledge in the art will appreciate that other components will be included in order to operate a commercially feasible device; however these are not depicted herein for simplicity and clarity of illustration.

The client device, in its embodiment as a mobile device, includes a Mobile Operating System (OS) which can be a proprietary OS developed and sold by a provider, such as iOS developed by Apple Inc., and Android OS developed by Google. The Mobile OS contains operating system features, in addition to other features common to smartphones, such as GPS, camera, music player, Bluetooth, and the like.

The Mobile OS will also include various apps, some of which are supplied “out of the box” (included with the device), and some of which are later acquired by the user. The apps can include, for example, XMPP, GPS, and the like. Future marketing and development will decide in which manner the service app will be provided. The User Interface App is what determines how the user interacts with the device. The User Interface App determines the “look and feel” of the various apps and controls such things as touch screens, swipes, icon views, and the like.

The Server is a computing system which can be a stand-alone computer or networked into a larger system. For purposes of this disclosure, the Server is a networked computing device in communication with other network devices. It will be readily appreciated that the Server, as a computer, includes conventional hardware and functionality required to operate as a provider of the service. This hardware and functionality are well known to those with knowledge in the art and thus are simply represented in the diagram as Server Processing/Storage components.

The User Interface Engine provides the service's graphical user interface with which the user of the service engages. Interaction between the user and the service is directed by the User Interface Engine, such as providing screens, receiving input, providing pop-up windows, and the like. The user interface processing is preferably performed using technologies such as Java-based graphical user interface technologies, although other similar technologies now known or subsequently developed may be used for the processing.

The Register Engine handles the user registration and interfaces with the Monetization Engine for processing any fees or upgrades attached to the registration. The Register Engine also builds the user profile.

The various modules and steps described in the method, above, are preferably embodied as modules or “engines” of the system.

### Alternative Embodiments

The various modules of the system and method steps of the service may be carried out in other, alternative, embodiments. For example, the invention may be carried out as an embodiment that focuses on the use of the service by a dentist that want to use the system to provide aligners directly to their patients.

In another alternative embodiment, some patients are not looking to move teeth but are just searching for a solution to keep their teeth straight. This is called retention. An embodiment for the system and method can be configured to offer a retention solution for patients who have had orthodontics in the past and would like a reliable and cost-effective source for high-quality retainers. Modules of this embodiment may include:

1. An oral health exam at an affiliate dentist to ensure that retention is an ideal solution for this patient and no dental work needs to be completed before the scan.

2. A scan

3. Delivery of the first set and upon verification of fit, the additional 4 sets are mailed to the patient.

Retention Membership.

Retention Membership Program is only available to previous patients of the service. The patient can opt into the membership program after purchasing the initial retention program or after completing any Aligner Treatment. This is only available to patients using an existing retainer scan from the Service or from the last tray of Aligners. If a new scan is needed due to dental work or changes in dental work, an additional fee for the scan and digital retainer set-up is accessed. This includes a year's storage of the STL image (3D data file) in the cloud (on the system's remote storage module) and unlimited retainers, on command. Since this is made off an STL of a previously delivered and verified retainer scan, it will be directly mailed to the patient In other words, the doctor has already delivered this version of the retainer, these are just duplicate sets so it doesn't need to be delivered by the doctor. It can be mailed to the patient.

The doctor must accept the patient in the retention program and the terms of service.

The doctor gets a percentage of all membership fees and retainer fees, except shipping. The doctor essentially does nothing except sign-up to get this production.

Alternatively, for any other orthodontic patients from the doctor's patient base, if the doctor has a previous scan, the appropriate module of the system can be configured to accept the scan data. Otherwise the doctor will need to submit a new scan. Doctor should verify fit in person if it is a new scan.

In light of the foregoing description, it should be recognized that embodiments in accordance with the present invention can be realized in numerous configurations contemplated to be within the scope and spirit of the claims. Additionally, the description above is intended by way of example only and is not intended to limit the present invention in any way, except as set forth in the claims. Modifications and changes to the invention should be readily apparent to those having ordinary skill in the art, which modifications are intended to be within the spirit and scope of the invention as claimed. It is also understood that the foregoing description is illustrative of the present invention and should not be considered as limiting. Components from one embodiment can be combined with another embodiment and remain within the spirit and scope of the invention. Therefore, other embodiments of the present invention are possible without departing from the spirit and scope of the present invention.

1. A computer-implemented method of providing, fitting, and treating a patient with clear aligners, the computer-implemented method comprising:
providing a computer-implemented software application accessible by a user on an electronic device, the electronic device comprising a processor, a memory, and a GPS module;
querying by the computer-implemented software application of the GPS module for the user's geographic location;
identification by the computer-implemented software application of a required location parameter defined by the user's geographic location received from the GPS module;
querying by the computer-implemented software application of a database of available providers for a one or more available provider that match the required location parameter;
returning by the computer-implemented software application a display of the one or more available provider that match the required location parameter;
interfacing by the computer-implemented software application with a one or more practice management software for each of the one or more available provider in order to return a one or more available appointment dates and times;
coordination of scheduling of an appointment between the user and the provider via the computer-implemented software application;
confirmation via the computer-implemented software application of a one or more additional parameter;
providing via the computer-implemented software application a confirmation of the appointment and reminders to the user;
once the user has had the consultation with the provider, the provider, via a provider-side interface to a backend database of the invention, uploads pertinent data such as, but not limited to intraoral 3D scans, x-rays, photographs, and other information pertinent to the user/patient;
the application system, upon confirmation by the provider, provides the uploaded data to a manufacturer; and
the manufacturer, accessing the system, utilizes the uploaded data to create the clear aligners and sends them directly to the Provider/doctor to be delivered to the patient;
whereby the patient receives customized clear aligners and a treatment plan with the convenience of a direct to consumer DIY model but with the oversight of a dentist or orthodontist.

2. The method of claim 1, further comprising a subscription model whereby a provider may pay for a level of service and, in return, receive marketing, referrals, hardware, and custom-programmed treatment tools.

3. A system adapted such that a patient receives customized clear aligners and a treatment plan with the convenience of a direct to consumer DIY model but with the oversight of a dentist or orthodontist, the system comprising:
a network;
a specially-programmed back-end computer having a processor, a memory, and a database, the specially-programmed computer configured to communicate over the network;
a specially-programmed user-facing computer having a processor, a memory, and a GPS module, the specially-programmed user-facing computer configured to communicate over the network; and
a computer-implemented software program configured to:
identify and attract interested patients;
efficiently match patients with a local dentist or orthodontist;
handle the process via a network-connected application; and
require that the doctor or orthodontist see, treat, and administer the customized clear aligners with the patient;
whereby, the patient receives customized clear aligners and a treatment plan with the convenience of a direct to consumer DIY model but with the oversight of a dentist or orthodontist.

4. A system through which a clear aligner delivery method is implemented, the system comprising:
s hosting zone;
a server;
a client device;
a database coupled with the server;
a digital intraoral scanner; and
an application gateway facilitating communication between the client device and the server, whereby the application gateway is configured to:
provide a user interface to guide a user through a one or more process steps;
provide a one or more software module configured to access, modify, and write to the database coupled with the server; and
transform a one or more atoms of data in the database coupled with the server.

5. The system of claim 4 where the one or more process steps is chosen from one or more of the following:
salesperson enters provider lead into crm and follows sales process to close;
salesperson sends provider online signup forms for affiliate doctor agreement via portal;
provider signs and executes affiliate doctor agreement via online portal, submits payment of network fee, and completes all requirements;
the application gateway sends annual renewal reminder and process payment of network fee;
provider support verifies all data and activates new customer;
provider support performs remote training and provides resources to provider;
provider configures account and office settings, hours, and availability through portal;
the application gateway sets provider as ready to accept patients and adds to database of potential providers for patients;
patient interacts with marketing or visits website;
the application gateway provides patient list of potential providers based on patient's location data;
patient schedules initial scan appointment at doctor's office via online booking page/app;
the application gateway notifies patient immediately that appointment will be confirmed within a specified time frame, such as 24 hours, and provides patient informed consent and patient release to comply with HIPPA;
the application gateway links with doctor's practice management system for scheduling of appointments;
the application gateway coordinates with selected provider's calendar and enters appointment in provider's calendar and confirms appointment with patient;
alternatively, patient dials call center through the application gateway and schedules initial scan via phone;
customer service schedules patient's tentative initial scan appointment pending provider confirmation;
customer service or the application gateway sends “welcome” email with financing prequalification and lets patient know appointment is pending confirmation by provider, or alternatively, the application gateway will generate “welcome intro” email;
patient confirms with their insurance company to determine if they prequalify for insurance coverage;
customer service assists patient with forms to file their claim and handling of reimbursements directly with insurance company after purchase of the aligners;
options for credit card payment and financing are explained and offered later when treatment plan selected;
the application gateway offers a prequalification link to third-party financing and patient can enter estimated financing amount and finance company does a ‘soft pull’ credit check which does not affect patient's credit score, to provide a payment estimate subject to credit and apr;
customer service calls each provider to confirm dates and times requested;
the information is then given to the doctor's office per the patient's questionnaire and personal info submission in the booking system which confirms patient's appointment with provider through the application gateway or phone call;
the application gateway alerts customer service for all new appointment requests daily;
provider confirms patient's appointment via the application gateway or phone call with customer service representative;
the application gateway sends booking confirmation email and patient visits provider's office on appointment date for initial screening and scan;
the application gateway sends a reminder email, for example, 12 hours prior to appointment;
provider performs exam to determine patient is viable candidate for aligners and, if determined to be viable, takes intraoral 3d scan+x-rays+photographs patient for ‘before’ images;
doctor discusses additional services ipr (inter proximal reduction), attachments, etc. . . . that are needed for proper aligner treatment and satisfactory results;
if patient does not pass exam or patient simply is not a candidate due to age or other matters, then customer service sends email to patient about future treatment opportunities once their issue has been addressed and resolved;
if patient cancels/no show, then app/customer service sends “what happened?” email to patient and offers a second consultation booking with a link to booking system attached;
if patient passes exam, then in the provider's office, if doctor and patient agree, a follow up appointment is scheduled to review treatment plan and package selection;
if patient selects ‘complete solution’ option which is most comprehensive treatment plan a follow up appointment is not needed, the new appointment date and time is directly loaded into provider's practice management system/calendar;
provider inputs treatment requirements, scan files, x-rays, and images into the application gateway and the application gateway submits treatment plan requirements to treatment planner;
treatment planner creates multiple treatment plan options if needed based on provider requirements and product selection and revises plan as requested by provider based on patient feedback;
provider accepts treatment plan options (move forward to order)—or—requests revisions to treatment plan (send revisions to treatment planner);
patient selects treatment plan from options based on level of correction patient desires and budget and the application gateway notifies provider of treatment plan selected;
provider initiates the application gateway to send unique payment portal link to patient;
patient selects from credit card or financing options and completes payment wizard in the application gateway;
if selected, financing application is sent to 3rd party financing company through the application gateway;
the finance company is directly connected with the application gateway for application submission and decision and all required notices required for financing and finance company provides decision on credit application within 60 seconds;
financing company notifies patient directly through app of financing decision and, if approved, patient is sent “congratulations” email through the application gateway confirming financing approval, and, if not approved, customer service works with patient for alternative form of payment;
on confirmation of payment, the application gateway sends selected treatment plan and all necessary files for patient's case via portal to manufacturer;
manufacturer produces aligners and provides status updates via the application gateway;
patient receives confirmation from the application gateway that aligners are in production;
manufacturer ships aligners and tracking information is entered into the application gateway and sent to provider and patient;
provider receives aligners from manufacturer and inspects aligners on arrival;
provider confirms receipt and inspection of aligners through the application gateway;
provider schedules appointment with patient;
patient is notified through the application gateway that aligners have been delivered to doctor and if provider has not scheduled appointment, provides a link to schedule appointment;
patient visits provider's office on appointment date for aligner fitting and initiation of treatment;
provider checks fit, adds attachments/engagers or ipr as needed, and instructs patient on proper use and care;
on receipt of funds from financing company, merchant bank for credit card payment, or paypal payment, initiate initial payment via ACH to provider through the application gateway;
provider schedules via the application gateway regular follow-up appointments with patient throughout treatment until completion;
patient visits provider's office at regular intervals throughout treatment until completion as scheduled through the application gateway patient treatment is monitored and supervised by the provider to ensure teeth in both arches straight and that teeth and both arches are in the proper occlusion or the best occlusal position possible for patient;
customer service follows up with patient via the application gateway or phone call to check up on progress periodically to ensure case is progressing per treatment plan;
patient visits provider's office upon completion of orthodontic treatment;
provider inspects results, issues retainers, removes attachments/engagers, takes finishing photographs, and sends patient final sign-off forms through the application gateway provides initial retainers and orders custom retainers as needed for patient to be delivered by provider at office appointment scheduled through the application gateway;
patient signs release/sign-off forms, customer satisfaction survey, concluding treatment executes electronically in office or through the application gateway;
patient receives retainers;
customer service congratulate patient on successful treatment and provide incentive for referral, whereby patient treatment is now complete;
initiate payment of remaining balance of payment to provider through the application gateway; or
the application gateway certifies all payments made and disbursed to provider, all patient closing forms signed and filed and then archives patient case and case remains available for access and follow up by provider and service, whereby provider treatment is now complete.
